(19) United States
(12) Patent Application Publication
(10) Pub. No.: US 2012/0087888 A1
(43) Pub. Date: Apr. 12, 2012

(54) Bio-Mechanical Stimulation Of Collagen Synthesis In Skin Cells And Reduction Of Appearance Of Fine Lines And Wrinkles On The Skin

(76) Inventors: Nadine A. Pernodet, Huntington Station, NY (US); Donald F. Collins, Plainview, NY (US); Jean Harry Xavier, Holbrook, NY (US); Phillip Cummins, Livingston, NJ (US); Christina G. Fthenakis, Dix Hills, NY (US); William Robert Bickford, Ronkonkoma, NY (US); Lenny Slutsky, Dunedin, FL (US)

(21) Appl. No.: 13/082,889

(22) Filed: Apr. 8, 2011

Related U.S. Application Data
(60) Provisional application No. 61/322,956, filed on Apr. 12, 2010.

(51) Int. Cl. A61K 8/81; A45D 34/04; A45D 40/26; A61Q 19/08

(52) U.S. Cl. 424/78.03; 401/292; 401/34

(57) ABSTRACT

The present invention achieves bio-mechanical stimulation of collagen synthesis in skin cells and reduction of the appearance of fine lines and wrinkles on the skin by applying a polymeric composition onto the skin in a manner so as to create surface tension that mimics the natural mechanical tension found in youthful skin.

### CROSS-REFERENCE TO RELATED APPLICATIONS

The present application claims priority from U.S. Provisional Patent Application Ser. No. 61/322,956, filed Apr. 12, 2010.

### FIELD OF THE INVENTION

The present invention relates to a method for applying a polymeric composition onto the skin in a manner so as to create surface tension across the skin that mimics the natural mechanical tension found in youthful skin, thereby bio-mechanically stimulating collagen synthesis and reducing the appearance of fine lines and wrinkles on the skin.

### BACKGROUND OF THE INVENTION

Mechanical forces are important regulators for maintaining proper functionality of the skin. Skin cells sense strains (i.e., deformation) in the extracellular matrix (ECM) caused by mechanical stresses and translate this information into adaptive responses, such as, for example, increase or decrease in the protein production, by a feedback and response mechanism. There are constant communications between the skin cells and the ECM, and cellular responses to mechanical strains or stresses are generated within sub-seconds through a 3-step process, which includes mechano-sensing, mechano-transduction, and mechano-response. When an external tension is applied to the ECM, it causes exposure of biologically active cryptic sites in the ECM, which in turn triggers ECM matrix assembly in the extracellular environment. Specifically, the ECM matrix assembly involves recruitment of various matrix proteins at the affected ECM sites, recruitment and translation of integrins, remodeling/stretching of the matrix, force-induced switching of matrix functionalities, and the like. The external tension also affects the adhesion sites between the ECM and the skin cell, which leads to structural reorganization of cytoskeleton and propagation of signals from the adhesion sites to the nucleus of the cell. In response, the cell alters the protein expression levels and adjusts the cellular functions to accommodate changes in the extracellular environment.

As humans age, the skin gradually loses its mechanical tension. Collagen fibers in the skin are responsible for providing and maintaining the mechanical tension, which in turn dictates fibroblast functionalities and vice versa. Existence of a sufficient amount of mechanical tension or stretch is critical for maintaining balanced production of proteins and proteases by the fibroblast cells. However, the lack of mechanical tension in the aged skin causes the fibroblast cells to collapse. The collapsed fibroblast cells tend to produce less collagen and more collagenases, which in turn leads to further reduction of mechanical tension in the skin. In other words, this is a vicious cycle that speeds up the process of skin aging.

It is an object of the present invention to provide a method for bio-mechanically stimulating collagen synthesis and reducing the appearance of fine lines and wrinkles on the skin, by applying a mechanical tension across the skin to mimic the natural mechanical tension found in youthful skin.

### SUMMARY OF THE INVENTION

The present invention provides a method for bio-mechanically stimulating collagen synthesis in skin cells and reducing the appearance of fine lines and wrinkles in skin, comprising:
(a) forming a polymeric composition comprising a first polymer and a second polymer that are dissolved or dispersed in a solvent system containing one or more solvents, wherein the first polymer is an anionic polymer capable of contracting upon solvent evaporation, wherein the second polymer is a cationic polymer capable of forming a polymeric complex with the first polymer and simultaneously binding to skin surface;
(b) applying the polymeric composition to a first region and a second region on the skin, wherein the first and second regions are spaced apart from each other by a pre-determined distance, with at least one fine line or wrinkle therebetween; and
(c) drying the applied polymeric composition at the first and second regions on the skin, so as to evaporate the one or more solvents in the solvent system and cause contraction of the polymeric composition at the first and second regions,
   wherein the contraction creates a mechanical tension across the skin surface between the first and second regions, which functions to bio-mechanically stimulate collagen synthesis in skin cells and reduce the appearance of fine lines or wrinkles on the skin.

The present invention also provides a cosmetic device comprising:
a housing comprising a first compartment filled with a polymeric composition comprising a first polymer and a second polymer that are dissolved or dispersed in a solvent system containing one or more solvents, wherein the first polymer is an anionic polymer capable of contracting upon solvent evaporation, wherein the second polymer is a cationic polymer capable of forming a polymeric complex with the first polymer and simultaneously binding to skin surface; and
a first applicator head located at one end of the housing and in fluid communication with the first compartment, wherein the first applicator head is arranged and constructed for dispensing the polymeric composition onto a skin surface to form an elongated strip having a width ranging from about 2 mm to about 2 cm.

In a preferred but not necessary embodiment of the present invention, the housing further comprises a second compartment filled with a cosmetic composition comprising at least one active ingredient capable of biologically stimulating collagen synthesis in skin cells and reducing the appearance of fine lines and wrinkles on the skin, and the device further comprises a second applicator head located at the other, opposite end of the housing and in fluid communication with the second compartment. The second applicator head is arranged and constructed to dispense the cosmetic composition onto a skin surface in form of a line having a width ranging from about 0.1 mm to about 2 mm.

The present invention further provides a topical composition comprising:
(a) from about 20 wt % to about 40 wt % of a first polymer selected from the group consisting of sodium polystyrene sulfonate, styrene/acrylates/ammonium methacrylate copolymer, vinyl caprolactam/vinylpyrrolidone/dimethylaminoethylmethacrylate copolymer, vinylpyrrolidone/dimethylaminopropylmethacrylamide copolymer, vinylpyrrolidone/dimethylaminoethylmethacrylate copolymer, and dimethylacrylamide/acrylic acid/polystyrene ethyl methacrylate copolymer;
(b) from about 1 wt % to about 20 wt % of a second polymer selected from the group consisting of polyphosphorylcholine butylmethacrylate copolymer, vinylpyrrolidone/dimethylaminopropylmethacrylate/methylaminopropyldimethyl methacrylate copolymer, vinylpyrrolidone/methacrylamidopropyl trimethylammonium chloride copolymer, vinylpyrrolidone/dimethylaminoethyl methacrylate copolymer, and quaternized hydroxyethylcellulose/hyaluronic acid complex;
(c) from about 0.1 wt % to about 1 wt % of a preservative; and
(d) from about 40 wt % to about 80 wt % of water.

Other aspects and objectives of the present invention will become more apparent from the ensuring description, examples, and claims.

### DEFINITION

The term “percentage” or “%” as used herein in connection with the amount or concentration of an ingredient or component in a composition refers to the percentage by total weight of the final composition, unless otherwise specified.

The term “substantially parallel” as used herein refers to a maximum deviation of ±30° from the parallel direction.

The term “stretch” as used herein refers to the percentage of skin deformation caused by application of the polymeric composition of the present invention onto the skin and subsequent drying thereof, as measured by the modified Digital Image Speckle Correlation (DISC) technique as described in U.S. Patent Application Publication No. 2009/0022665A1, the content of which is incorporated herein by reference in its entirety for all purposes.

### BRIEF DESCRIPTION OF THE DRAWINGS

FIG. 1 is a bar chart comparing the amounts of type I collagen produced per cell by three different types of dermal fibroblast cells obtained from individuals of different ages (neonatal, 24 years old, and 45 years old, respectively) with or without application of a static linear strain after 24 hours of growth in vitro.

FIG. 2 is a bar chart comparing the amounts of fibronectin produced per cell by three different types of dermal fibroblast cells obtained from individuals of different ages (neonatal, 24 years old, and 45 years old, respectively) with or without application of a static linear strain after 24 hours of growth in vitro.

FIG. 3 is a bar chart comparing the amounts of laminin produced per cell by four different types of dermal fibroblast cells obtained from individuals of different ages (neonatal, 24 years old, 45 years old, and 68 years old, respectively) with or without application of a static linear strain after 24 hours of growth in vitro.

FIGS. 4A and 4B are schematic diagrams illustrating how a polymeric composition of the present invention is applied to a wrinkle on the skin to create a mechanical tension across the skin surface for bio-mechanically stimulating collagen synthesis in skin cells and reducing the appearance of the skin wrinkle.

FIG. 5 is a schematic diagram showing how a polymeric composition of the present invention can be applied to the facial area of a user for bio-mechanical treatment of typical facial lines and wrinkles.

FIGS. 6A and 6B are side and top views of an exemplary cosmetic device of the present invention for applying a polymeric composition onto the skin to form elongated polymeric strips.

FIG. 7 is a side view of an exemplary cosmetic device of the present application with first and second applicator heads for applying a polymeric composition of the present application and an additional cosmetic composition onto the skin.

### DETAILED DESCRIPTION OF THE INVENTION AND PREFERRED EMBODIMENTS THEREOF

In order to evaluate the impact of deformation caused by application of external mechanical force or strain on the protein expression levels of skin cells growing in vitro, inventors of the present application employed a FlexCell® FX-5000™ system manufactured by FlexCell International Corp at Hillsborough, N.C. The FlexCell® FX-5000™ system is a computer-regulated bioreactor that uses vacuum pressure to apply cyclic or static strain to cells cultured on flexible-bottomed culture plates. A defined, controlled static or cyclic deformation can be applied to cells growing in vitro, and the degree of deformation is regulated by the vacuum force applied, which can yield up to 25% substrate elongation.

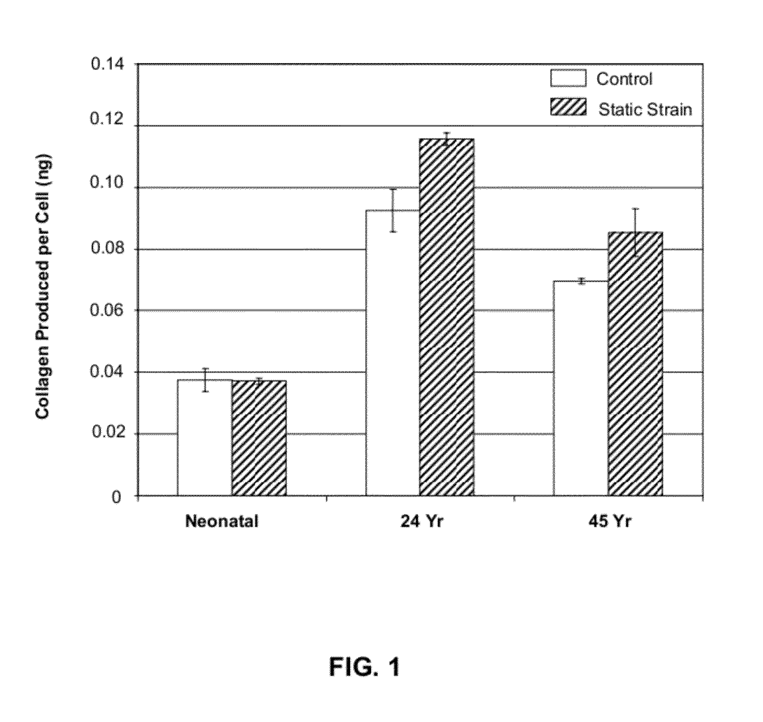

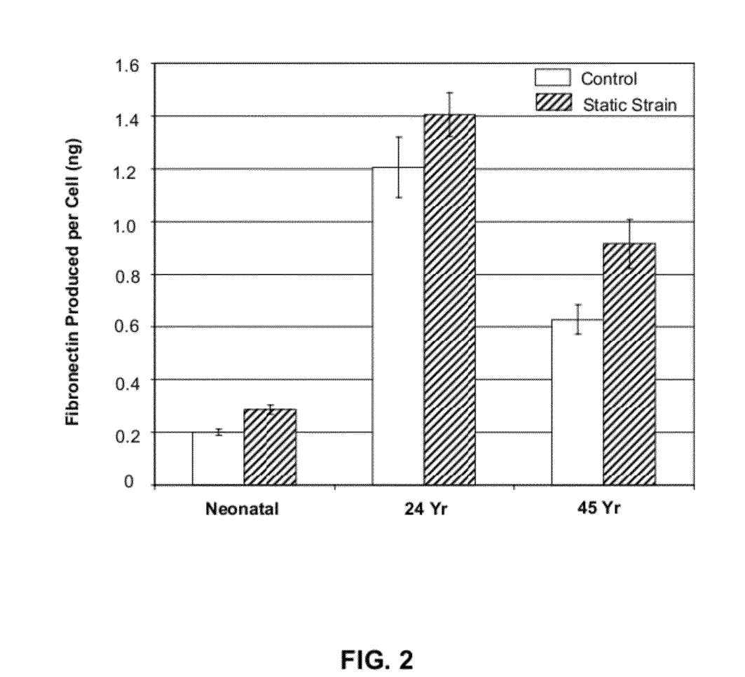

Three different types of dermal fibroblast cells, which included neonatal dermal fibroblast cells, dermal fibroblast cells obtained from a 24-year-old individual, and dermal fibroblast cells obtained from a 45-year-old individual, were tested to see the impact of externally applied strain on the protein expression levels in the cells obtained from individuals of different ages. All three types of dermal fibroblast cells were placed in the cell culture plates of the FlexCell® FX-5000™ system. The different types of dermal fibroblasts are grown and sub-cultured in Dulbecco's Modified Eagle Medium (Catalog number 11965, Invitrogen, Carlsbad, Calif.) supplemented with 10% Fetal Bovine Serum (Catalog number SH30071.03, Hyclone, Logan Utah) and 1% Penicillin (5000 IU/ml)/Streptomycin (5000 μg/ml) (Catalog number 30-001-CI, Mediatech, Manassas, Va.). The cells are grown at 37° C. in a 100% humidified atmosphere containing 5% CO₂. A vacuum force was applied to each culture plate to produce a static linear strain of about 10% substrate elongation for about 24 hours. Subsequently, the amount of type I collagen and fibronectin produced per cell was measured for each type of dermal fibroblasts and then compared with that produced by the control cells (i.e., the same type of dermal fibroblasts grown for 24 hours but without the strain). The measurement results were illustrated in FIGS. 1 and 2, which indicate that application of the static linear strain had little or no effect on the type I collagen and fibronectin expression levels in the neonatal dermal fibroblast cells, but it significantly increased the type I collagen and fibronectin expression levels in the dermal fibroblasts obtained from the 24-year-old and 45-year-old individuals.

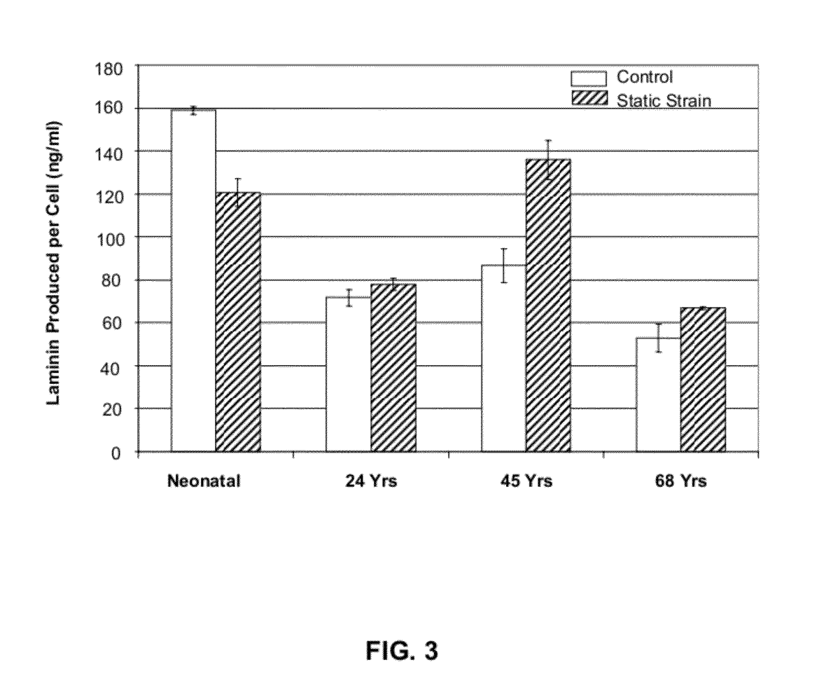

Similar tests as described hereinabove were conducted to measure the impact of static linear strain on the laminin expression level in dermal fibroblasts. Specifically, four different types of dermal fibroblast cells, which included neonatal dermal fibroblast cells and dermal fibroblast cells obtained from a 24-year-old individual, a 45-year-old individual, and a 68-year-old individual, were placed in the cell culture plates of the FlexCell® FX-5000™ system. A vacuum force was applied to each culture plate to produce a static linear strain of about 10% substrate elongation for about 24 hours. Subsequently, the amount of laminin produced per cell was measured for each type of dermal fibroblasts and was then compared with that produced by the control cells (i.e., the same type of dermal fibroblasts grown for 24 hours but without the strain). The measurement results were illustrated in FIG. 3, which indicate that application of the static linear strain had either little effect or even negative effect on laminin expression level in the more youthful cells (i.e., the neonatal and the 24-year-old dermal fibroblast cells), but it significantly increased the laminin expression level in the more aged cells (i.e., the 45-year-old and the 68-year-old dermal fibroblasts).

Collagen, fibronectin, and laminin are important proteins responsible for supporting the structural integrity and cellular functionality of the skin cells and improving the elasticity and firmness of the skin. Based on the experimental results described hereinabove, inventors of the present invention believe that application of an external strain or tension across the skin surface can effectively stimulate synthesis of these important proteins by the skin cells, which in turn will improve the elasticity and firmness of the skin, reduce the appearance of fine lines and wrinkles on the skin, and render a more youthful appearance of the user.

Such an external strain or tension across the skin surface is achieved in the present invention through application of a polymeric composition to the skin, which is capable of contracting upon drying. Specifically, the polymeric composition of the present invention contains a first polymer and a second polymer either dissolved or dispersed in a solvent system containing one or more solvents. The first polymer is an anionic polymer capable of contracting upon evaporation of the one or more solvents, while the second polymer is a cationic polymer capable of forming a polymeric complex with the first polymer and simultaneously binding to the skin surface. In combination, the first and second polymers form a polymeric network that binds well to the skin surface and is also capable of contracting upon solvent evaporation to pull or stretch the skin.

The solvent system as described hereinabove can include any solvent or solvents suitable for use in cosmetic or skin care products. Suitable solvents that can be used in the polymeric composition of the present invention include, but are not limited to: water; C1-C4 alcohols such as ethanol, propanol, isopropanol; polyols and polyol ethers such as carbitols, 2-butoxyethanol, propylene glycol, propylene glycol monomethyl ether, diethylene glycol monoethyl ether, monomethyl ether, butylene glycol, hexylene glycol, glycerol, ethoxy glycol, ethoxy diglycol; propylene carbonate; and mixtures thereof. In a preferred but not necessary embodiment of the present invention, the solvent system contains water and optionally one or more water-miscible solvents. In a more preferred embodiment, the solvent system consists essentially of water.

The amount of solvent(s) contained by the polymeric composition of the present invention may range from about 1% to about 99% by total weight of the composition, preferably from about 10% to about 90% by weight, and more preferably from about 40% to about 80% by weight.

The first polymer as described hereinabove is preferably a water-soluble or water dispersible anionic polymer, such as, for example, sodium polystyrene sulfonate, which is commercially available from Akzo Nobel Surface Chemistry LLC (Chicago, Ill.) under the trademark Flexan® II; styrene/acrylate/ammonium methacrylate copolymer, which is commercially available from Interpolymer Corporation (Louisville, Ky.) under the trademark Syntran® PC5100NP; vinyl caprolactam/vinylpyrrolidone/dimethylaminoethylmethacrylate copolymer, which is commercially available from International Specialty Products (Wayne, N.J.) under the trademarks Advantage® S and Gaffix® VC-713; vinylpyrrolidone/dimethylaminopropylmethacrylamide copolymer, which is commercially available from International Specialty Products (Wayne, N.J.) under the trademark Styleze® CC-10; vinylpyrrolidone/dimethylaminoethylmethacrylate copolymer, which is commercially available from International Specialty Products (Wayne, N.J.) as Copolymer 958; and a dimethylacrylamide/acrylic acid/polystyrene ethyl methacrylate copolymer, which is commercially available from Polytherapeutics, Inc. (New Brunswick, N.J.) under the trademark PharmaDur®.

The amount of the first polymer as contained by the polymeric composition of the present invention may range from about 1% to about 60% by total weight of the composition, preferably from about 10% to about 50% by weight, and more preferably from about 20% to about 40% by weight.

The second polymer as described hereinabove is preferably a water-soluble or water dispersible cationic polymer, such as, for example, polyphosphorylcholine butylmethacrylate copolymer, which is also referred to as “polyquaternium-51” and is commercially available from NOF Corporation (Tokyo, Japan) under the trademarks Lipidure®-PMB and Lipidure®-Ph10; vinylpyrrolidone/dimethylaminopropylmethacrylate/methylaminopropyldimethylmethacrylate copolymer, which is also referred to as “polyquaternium-55” and is commercially available from International Specialty Products (Wayne, N.J.) under the trademark Styleze® W-20; vinylpyrrolidone/methacrylamidopropyl trimethylammonium chloride copolymer, which is also referred to as “polyquaternium-28” and is commercially available from International Specialty Products (Wayne, N.J.) under the trademark Gafquat® HS-100; vinylpyrrolidone/dimethylaminoethyl methacrylate copolymer, which is also referred to as “polyquaternium-11” and is commercially available from International Specialty Products (Wayne, N.J.) under the trademark Gafquat® 755N; an association complex of quaternized hydroxyethylcellulose (also referred to as “polyquaternium-24”) and high molecular weight hyaluronic acid, which is commercially available from Amerchol Corporation (Bound Brook, N.J.) under the trade name BIOCARE Polymer HA-24.

The amount of the second polymer as contained by the polymeric composition of the present invention may range from about 0.1% to about 50% by total weight of the composition, preferably from about 1% to about 20% by weight, and more preferably from about 2% to about 15% by weight.

The polymeric composition of the present invention may also contain a preservative, preferably a water-soluble preservative, such as phenoxyethanol, potassium sorbate, imidazolidinyl urea, p-hydroxy benzoate, esters of p-hydroxybenzoic acid, CTFA designation parabens, ethylhexylglycerin, caprylyl glycol/phenoxyethanol/hexylene glycol, etc. Other preservatives suitable for use in the polymeric compositions of the present invention are disclosed in the International Cosmetic Ingredient Dictionary and Handbook, twelfth edition, 2004, the entire disclosure of which is herein incorporated by reference.

The amount of the preservative as contained by the polymeric composition of the present invention may range from about 0.001% to about 10% by total weight of the composition, preferably from about 0.01% to about 5% by weight, and more preferably from about 0.1% to about 1% by weight.

In a preferred, but not necessary, embodiment of the present invention, the polymeric composition as described hereinabove comprises from about 20 wt % to about 40 wt % of the first polymer, from about 1 wt % to about 20 wt % of the second polymer, from about 0.1 wt % to about 1 wt % of a preservative, and from about 40 wt % to about 80 wt % of water. More preferably, the first polymer is sodium polystyrene sulfonate; the second polymer is polyphosphorylcholine butylmethacrylate copolymer; and the preservative is phenoxyethanol. In a still more preferred embodiment of the present invention, the polymeric composition comprises from about 25 wt % to about 30 wt % of sodium polystyrene sulfonate, from about 8 wt % to about 12 wt % of polyphosphorylcholine butylmethacrylate copolymer, from about 0.3 wt % to about 0.8 wt % of phenoxyethanol, and from about 55 wt % to about 70 wt % of water. Most preferably, the polymeric composition of the present invention consists essentially of the above-described ingredients.

The polymeric composition of the present invention may further contain one or more skin care active ingredients or skin care actives known in the art. The term “skin care active ingredients” or “skin care actives” as used herein refers to agents that provide benefits to the skin rather than merely improving the physical characteristics of the topical composition. For example, the polymeric composition may comprise anti-aging agents that are capable of protecting the skin against photo- or chrono-aging by scavenging free radicals, preventing lipid peroxidation, inactivating lipoxygenase, inhibiting undesired enzymatic activities, and stimulating collagen synthesis. The polymeric composition may also include anti-acne agents, enzyme-inhibiting agents, collagen-stimulating agents, sunscreen agents, antioxidant, exfoliants, agents for the eradication of age spots, keratoses and wrinkles, analgesics, anesthetics, antibacterials, antiyeast agents, antifungal agents, antiviral agents, antidandruff agents, antidermatitis agents, antipruritic agents, antiemetics, anti-inflammatory agents, antihyperkeratolytic agents, antiperspirants, antipsoriatic agents, antiseborrheic agents, antiwrinkle agents, antihistamine agents, skin lightening agents, depigmenting agents, vitamins, corticosteroids, self-tanning agents, hormones, retinoids such as retinoic acid and retinol, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, topical steroids such as hydrocortisone, hydrocortisone 21-acetate, hydrocortisone 17-valerate, and hydrocortisone 17-butyrate, betamethasone valerate, betamethasone diproprionate, benzoyl peroxide, crotamiton, propranolol, promethazine, vitamin A palmitate, vitamin E acetate and mixtures thereof.

The above-described skin care active ingredients are only optional components of the polymeric composition of the present invention and may be omitted from such composition without materially affecting the desired function of the polymeric composition.

The polymeric composition of the present application may further comprise substances commonly used in topical or skin care compositions, which include, but are not limited to: moisturizing agents, astringent agents, chelating agents, surfactants, emollients, stabilizers, thickeners, humectants, pigments, etc. Such substances should be present only in amounts that do not affect the ability of the polymeric composition to bind to the skin surface and to contract upon solvent evaporation.

For example, emollients which may be used in the polymeric composition of the present invention include, but are not limited to: stearyl alcohol, cetyl alcohol, oleyl alcohol, isocetyl alcohol, fatty alcohols, propane-1,2-diol, butane-1,3-diol, octadecan-2-ol, glyceryl monostearate, isopropyl isostearate, stearic acid, isostearic acid, isocetyl stearate, isopropyl stearate, butyl stearate, isopropyl laurate, hexyl laurate, decyl oleate, isobutyl palmitate, cetyl palmitate, isopropyl palmitate, palmitic acid, dimethylpolysiloxane, glyceryl monoricinoleate, di-n-butyl sebacate, isopropyl myristate, butyl myristate, myristyl myristate, isopropyl linoleate, lauryl lactate, myristyl lactate, polyethylene glycol, triethylene glycol, lanoline, acetylated lanolin, sesame oil, coconut oil, arrachis oil, castor oil, mink oil, mineral oil, and petroleum. The emollient, if present in the compositions of the present invention, is preferably present in a total amount from about 1% to about 20%, and more preferably from about 5% to about 15% by total weight of the composition.

Further, any suitable thickening agent commonly used in cosmetic and personal care products can be added to the polymeric compositions of the present invention to improve the viscosity of the compositions. Preferably, the thickening agent includes, but is not limited to: starch, gum (such as gum arabic), clay (such as kaolin), hydrated aluminum silicate, magnesium aluminum silicate, fumed silica, polyacrylic acid, hydroxyethylcellulose, carboxyvinyl polymer, sodium carboxymethyl cellulose or other cellulose derivatives, ethylene glycol monostearate and sodium alginates. The thickening agent, if present in the compositions of the present invention, is preferably present in a total amount from about 1% to about 20%, and more preferably from about 5% to about 15% by total weight of the composition.

Humectants which may be used include, but are not limited to: polyhydric alcohols including glycerol, polyalkylene glycols, and alkylene polyols and mixtures thereof, hyaluronic acid, urea, glycerin, sorbitol, sodium 2-pyrrolidone-5-carboxylate, soluble collagen, dibutylphthalate and gelatin. The humectant, if present in the compositions of the present invention, is preferably present in a total amount from about 1% to about 20%, and more preferably from about 5% to about 15% by total weight of the composition.

The polymeric composition of the present invention may also contain additional cosmetic ingredients that add to the aesthetics of performance of the present invention. For example, pigments, powders and fragrances may be used to increase the aesthetic appeal of the present invention. Pigments can be selected from cosmetically acceptable inorganic and organic pigments, such as those disclosed in the International Cosmetic Ingredient Dictionary and Handbook, twelfth edition, 2004. Suitable inorganic pigments may include iron oxides, titanium dioxide, zinc oxide, metal silicates (such as micas, talc, kaolin, etc.), bismuth oxychloride and the like. Suitable organic pigments may include barium lake, calcium lake, aluminum lake, zirconium lake, calcium lake, D&C and FD&C colors and lakes thereof. Powders that can be added into the topical composition of the present invention include chalk, talc, fuller's earth, colloidal silicon dioxide, sodium polyacrylate, tetra alkyl and/or trialkyl ammonium smectites, and chemically modified magnesium aluminum silicate. The topical composition of the present invention may optionally comprise a fragrance in an amount sufficient to make the composition more appealing to the consumer. The pigment(s), powders, or fragrance, if present in the compositions of the present invention, is preferably present in a total amount from about 1% to about 20%, and more preferably from about 5% to about 15% by total weight of the composition.

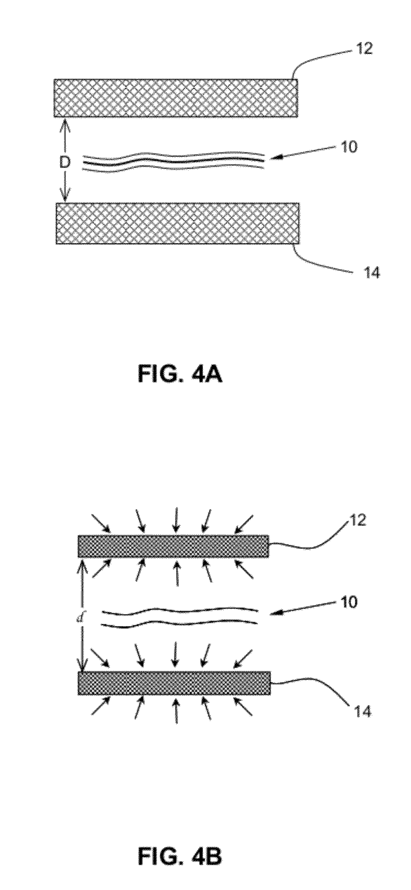

FIGS. 4A and 4B illustrate how such a polymeric composition can be used to create the desired strain or tension across the skin surface. As shown in FIG. 4A, a polymeric composition of the present application is applied to first and second regions on the skin to form first and second elongated polymeric strips 12 and 14. Preferably, but not necessarily, 12 and 14 are each characterized by a width ranging from about 2 mm to about 2 cm. The first and second elongated polymeric strips 12 and 14 are spaced apart from each other by a predetermined distance (D), with at least one fine line or wrinkle 10 therebetween. Preferably, but not necessarily, the elongated polymeric strips 12 and 14 extend along a direction that is substantially parallel to the fine line or wrinkle 10. FIG. 4B shows that upon subsequent drying and evaporation of solvent(s) from the polymeric composition, the elongated polymeric strips 12 and 14 contract to enlarge and the distance (d) therebetween, thereby pulling or stretching the skin to bio-mechanically stimulating collagen synthesis in the skin cells and reducing appearance of the fine line or wrinkle 10. Preferably, but not necessarily, the stretch of the skin is from about 5% to 20%, as measured by the modified Digital Image Speckle Correlation (DISC) technique described in U.S. Patent Application Publication No. 2009/0022665A1, the content of which is incorporated herein by reference in its entirety for all purposes.

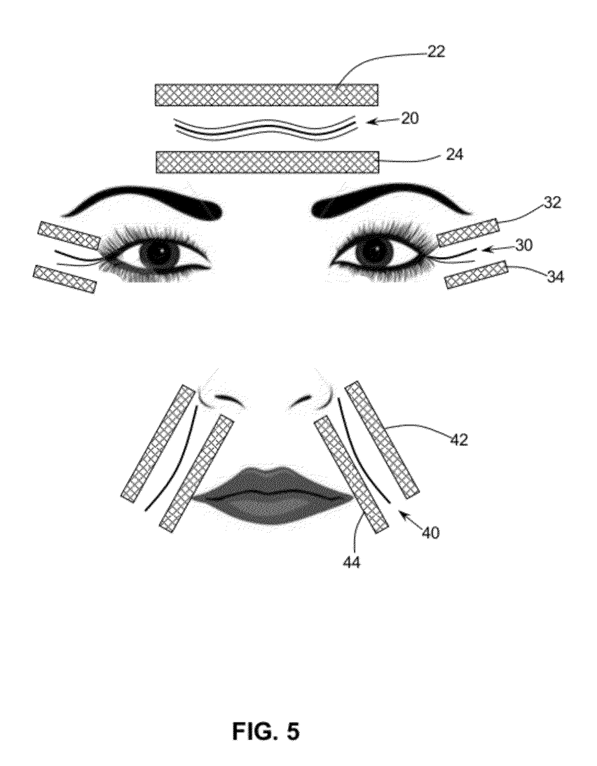

FIG. 5 illustrates how the polymeric composition of the present invention can be applied to various facial areas of a potential user. For example, the polymeric composition can be applied along a wrinkle 20 on the user's forehead to form two elongated polymeric strips 22 and 24, each of which is located at one side of the wrinkle 20 and is substantially parallel to wrinkle 20. The polymeric composition can also be applied along wrinkles 30 at the corner of the user's eye to form two elongated polymeric strips 32 and 34, each of which is located at one side of wrinkles 30 and are substantially parallel to wrinkles 30. The polymeric composition can further be applied along a wrinkle 40 around the user's mouth to form two elongated polymeric strips 42 and 44, each of which is located at one side of wrinkle 40 and are substantially parallel to wrinkle 40.

The specific methods of application in the present invention will depend on the ultimate intended use of the above-described polymeric composition. For example, the polymeric composition can be applied to the skin on an as-needed basis, to achieve immediate wrinkle reduction results (typically observable within five or ten minutes). Alternatively, it can be applied to the skin repeatedly according to a pre-set schedule to achieve long-term collagen synthesis boosting effect and wrinkle reduction effect. The polymeric composition of the present invention may be applied directly to clean skin, without application of any moisturizer, foundation, make-up, etc. Alternatively, the polymeric composition of the present invention can be applied over or under moisturizer, and optionally over or under foundation and/or make-up. The amount of the polymeric composition to be applied each time, the area of application, the duration of application, and the frequency of application can vary widely, depending on the specific need of the user. For example, the polymeric composition can be applied for a period of at least one month and at a frequency ranging from about once per week to about five times per day. For another example, the polymeric composition is applied for a period of about six months and at a frequency ranging from about three times a week to about three times per day, and preferably about once or twice per day.

The polymeric strips as described hereinabove can be readily formed using a cosmetic device containing a housing having a compartment filled with the polymeric composition described hereinabove, and an applicator head located at one end of the housing and in fluid communication with the liquid-filled compartment. The applicator head can have any shape or form and can be made of any material suitable for dispensing the polymeric composition onto a skin surface to form an elongated strip having a width ranging from about 2 mm to about 2 cm.

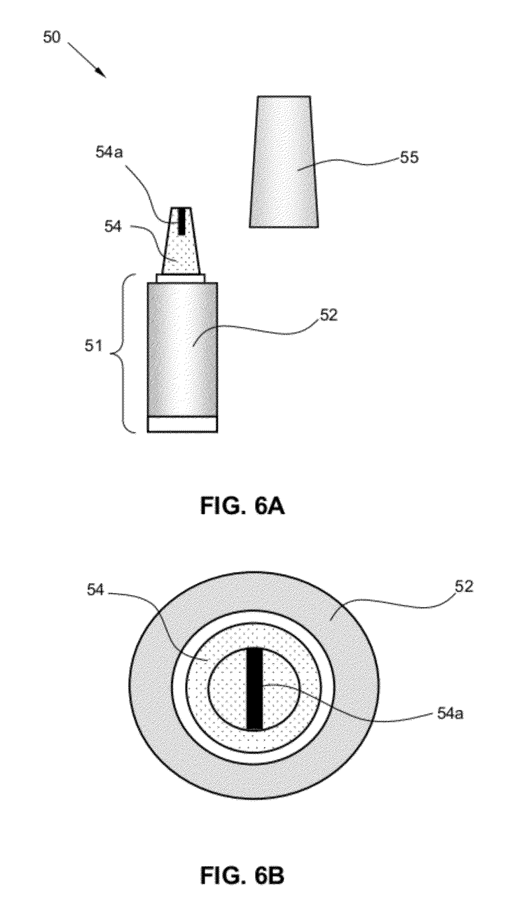

For example, FIGS. 6A and 6B show the side and top views of an exemplary cosmetic device 50, which comprises an elongated housing 51 that has a compartment 52 filled with the polymeric composition of the present invention. The cosmetic device 50 also contains an applicator head 54 that is located at one end of the elongated housing 51 and is in fluid communication with the liquid-filled compartment 52. At the tip of the applicator head 54, there is an elongated aperture 54a, through which the polymeric composition can be dispensed from the first compartment 52 to form elongated polymeric strips (not shown) on the skin surface. The width of the elongated polymeric strips so formed is defined by the length of the aperture 54a, which is preferably ranging from about 2 mm to about 2 cm. When not in use, the applicator head 54 is preferably covered by a cap 55 to avoid contamination or leakage.

In an alternative embodiment of the present application, a cosmetic composition comprising at least one active ingredient capable of biologically stimulating collagen synthesis in skin cells is further provided in addition to the polymeric composition described hereinabove, and such a cosmetic composition is directly applied to the fine lines and wrinkles on the skin to reduce the appearance thereof. Suitable active ingredients that can be used for forming such a cosmetic composition include, but are not limited to: vitamins A, C, and E and analogues and derivatives thereof (such as retinoic acid, retinal, retinol, retinyl acetate or retinyl palmitate, ascorbic acid, ascorbyl palmitate, magnesium ascorbyl phosphate, sodium ascorbyl phosphate, ascorbyl alpha- and beta-glucoside, tocopheryl, and tocopheryl acetate), aminoethyl compound, glucans (such as alpha-glucans and beta-glucans), certain growth factors (such as transforming growth factor or TGF beta), ginsenoside, certain hydrolyzed proteins (e.g., hydrolyzed milk or whey protein). Active ingredients which are frequently used to boost collagen synthesis also include peptide substances and derivatives thereof such as e.g. carnitine, carnosine, creatine, matrikine peptides (e.g. lysyl-threonyl-threonyl-lysyl-serine), peptide structures such as palmitoylated pentapeptides (e.g. Matrixyl from Sederma), and the oligopeptide with the trade name Vincipeptide (from Vincience, France). Moreover, compounds such as asiatic acid, madecassic acid, madecassoside, asiaticoside, plant extracts such as those from Aloe, Centella, and Plantago species, soy extract and soy isoflavones, extracts from Ginkgo biloba, niacinamide, astaxanthine, genistein, daidzein, rutin, chrysin, morin, betel nut alkaloids, forskolin, betulinic acid, glutamine, glycolic acid, collagen fragments, flavonoids, tannins and saponins such as those from Mimosa bark, Kudzu root or Scutellaria root, resveratrol and derivatives thereof, and water-soluble salts of aluminum (such as aluminum chloride), calcium, magnesium, and iron are used as collagen synthesis stimulators.

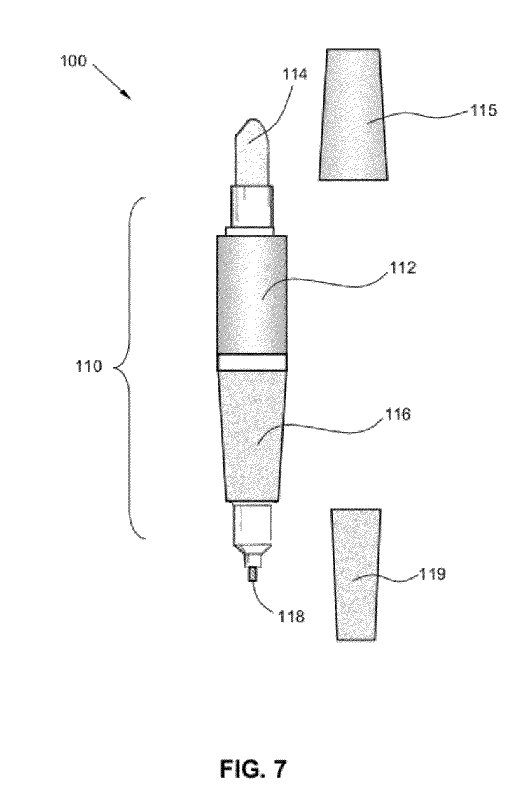

The cosmetic composition described hereinabove can be used in conjunction with the polymeric composition of the present invention to achieve both biological and bio-mechanical stimulation of the collagen synthesis. FIG. 7 shows the side-view of an exemplary cosmetic device 100 that can be used to conjunctively dispense the polymeric composition and the cosmetic composition for dual-action wrinkle treatment. Specifically, the cosmetic device 100 contains an elongated housing 110 having two liquid compartments 112 and 116. The first liquid compartment 112 is filled with the polymeric composition of the present invention as described hereinabove, while the second liquid compartment 116 is filled with the cosmetic composition described hereinabove. A first applicator head 114 is located at one end of the housing 110 and is in fluid communication with the first compartment 112. The first applicator head 114 can be formed of a porous material, such as felt or nylon fibers, for dispensing the polymeric composition onto a skin surface to form an elongated polymeric strip having a width ranging from about 2 mm to about 2 cm. A second applicator head 118 is located at the other, opposite end of the housing 110 and is in fluid communication with the second compartment 116. The second applicator head 118 can be formed as a pen tip for dispensing the cosmetic composition directly into a fine line or wrinkle in form of a line having a width ranging from about 0.1 mm to about 2 mm. When not in use, the applicator heads 114 and 118 are preferably covered by caps 115 and 119 respectively to avoid contamination or leakage.

Although the invention has been variously disclosed herein with reference to illustrative embodiments and features, it will be appreciated that the embodiments and features described hereinabove are not intended to limit the scope of the invention, and that other variations, modifications and other embodiments will suggest themselves to those of ordinary skill in the art. The invention therefore is to be broadly construed, consistent with the claims hereafter set forth.

What is claimed is:

1. A method for bio-mechanically stimulating collagen synthesis in skin cells and reducing the appearance of fine lines or wrinkles in skin, comprising:
(a) forming a polymeric composition comprising a first polymer and a second polymer that are dissolved or dispersed in a solvent system containing one or more solvents, wherein said first polymer is an anionic polymer capable of contracting upon solvent evaporation, wherein said second polymer is a cationic polymer capable of forming a polymeric complex with the first polymer and simultaneously binding to skin surface;
(b) applying the polymeric composition to a first region and a second region on the skin, wherein the first and second regions are spaced apart from each other by a predetermined distance, with at least one fine line or wrinkle therebetween; and
(c) drying the applied polymeric composition at the first and second regions on the skin, so as to evaporate the one or more solvents in the solvent system and cause contraction of the polymeric composition at the first and second regions,
wherein said contraction creates a mechanical tension across the skin surface between the first and second regions, which functions to bio-mechanically stimulate collagen synthesis in skin cells and reduce the appearance of fine lines or wrinkles on the skin.

2. The method of claim 1, wherein the solvent system comprises water.

3. The method of claim 2, wherein the first polymer is a water-soluble or water-dispersible anionic polymer selected from the group consisting of:
(i) sodium polystyrene sulfonate,
(ii) styrene/acrylates/ammonium methacrylate copolymer,
(iii) vinyl caprolactam/vinylpyrrolidone/dimethylaminoethylmethacrylate copolymer,
(iv) vinylpyrrolidone/dimethylaminopropylmethacrylamide copolymer,
(v) vinylpyrrolidone/dimethylaminoethylmethacrylate copolymer, and
(vi) dimethylacrylamide/acrylic acid/polystyrene ethyl methacrylate copolymer.

4. The method of claim 3, wherein the second polymer is a water-soluble or water-dispersible cationic polymer selected from the group consisting of:
(i) polyphosphorylcholine butylmethacrylate copolymer,
(ii) vinylpyrrolidone/dimethylaminopropylmethacrylate/methylaminopropyldimethyl methacrylate copolymer,
(iii) vinylpyrrolidone/methacrylamidopropyl trimethylammonium chloride copolymer,
(iv) vinylpyrrolidone/dimethylaminoethyl methacrylate copolymer, and
(v) quaternized hydroxyethylcellulose/hyaluronic acid complex.

5. The method of claim 1, wherein the polymeric composition comprises:
(a) from about 20 wt % to about 40 wt % of sodium polystyrene sulfonate;
(b) from about 1 wt % to about 20 wt % of polyphosphorylcholine butylmethacrylate copolymer;
(c) from about 0.1 wt % to about 1 wt % of a preservative; and
(d) from about 40 wt % to about 80 wt % of water.

6. The method of claim 1, wherein the polymeric composition is applied to the first and second regions on the skin in form of elongated polymeric strips extending along a direction that is substantially parallel to the at least one fine line or wrinkle.

7. The method of claim 6, wherein each of the elongated polymeric strips has a width ranging from about 2 mm to about 2 cm.

8. The method of claim 6, wherein the elongated polymeric strips contract upon solvent evaporation to create a stretch ranging from about 5% to about 20%.

9. The method of claim 1, further comprising applying to the at least one fine line or wrinkle a cosmetic composition comprising at least one active ingredient capable of biologically stimulating collagen synthesis in skin cells and reducing the appearance of fine lines and wrinkles on the skin.

10. The method of claim 9, wherein said at least one active ingredient is selected from the group consisting of vitamins A, C, and E and analogues and derivatives thereof, aminoethyl compound, glucans, growth factors, ginsenoside, hydrolyzed proteins, peptide substances or structures and derivatives thereof, collagen fragments, asiatic acid, madecassic acid, madecassoside, asiaticoside, plant extracts from Aloe, Centella, and Plantago species, soy extract and soy isoflavones, extracts from Ginkgo biloba, niacinamide, astaxanthine, genistein, daidzein, rutin, chrysin, morin, betel nut alkaloids, forskolin, betulinic acid, glutamine, glycolic acid, flavonoids, tannins, saponins, resveratrol and derivatives thereof, water-soluble salts of aluminum, calcium, magnesium, and iron, and mixtures thereof.

11. The method of claim 9, wherein said cosmetic composition is applied directly to the fine line or wrinkle in form of a line having a width ranging from about 0.1 mm to about 2 mm.

12. A cosmetic device comprising:
a housing comprising a first compartment filled with a polymeric composition comprising a first polymer and a second polymer that are dissolved or dispersed in a solvent system containing one or more solvents, wherein said first polymer is an anionic polymer capable of contracting upon solvent evaporation, wherein said second polymer is a cationic polymer capable of forming a polymeric complex with the first polymer and simultaneously binding to skin surface; and
a first applicator head located at one end of said housing and in fluid communication with said first compartment, wherein said first applicator head is arranged and constructed for dispensing the polymeric composition onto a skin surface to form an elongated strip having a width ranging from about 2 mm to about 2 cm.

13. The cosmetic device of claim 12, wherein the first polymer is a water-soluble or water-dispersible anionic polymer selected from the group consisting of sodium polystyrene sulfonate, styrene/acrylates/ammonium methacrylate copolymer, vinyl caprolactam/vinylpyrrolidone/dimethylaminoethylmethacrylate copolymer, vinylpyrrolidone/dimethylaminopropylmethacrylamide copolymer, vinylpyrrolidone/dimethylaminoethylmethacrylate copolymer, and dimethylacrylamide/acrylic acid/polystyrene ethyl methacrylate copolymer, and wherein the second polymer is a water-soluble or water-dispersible cationic polymer selected from the group consisting of polyphosphorylcholine butylmethacrylate copolymer, vinylpyrrolidone/dimethylaminopropylmethacrylate/methylaminopropyldimethyl methacrylate copolymer, vinylpyrrolidone/methacrylamidopropyl trimethylammonium chloride copolymer, vinylpyrrolidone/dimethylaminoethyl methacrylate copolymer, and quaternized hydroxyethylcellulose/hyaluronic acid complex, and wherein the solvent system comprises water.

14. The cosmetic device of claim 12, wherein the polymeric composition comprises:
(a) from about 20 wt % to about 40 wt % of sodium polystyrene sulfonate;
(b) from about 1 wt % to about 20 wt % of polyphosphorylcholine butylmethacrylate copolymer;
(c) from about 0.1 wt % to about 1 wt % of a preservative; and
(d) from about 40 wt % to about 80 wt % of water.

15. The cosmetic device of claim 12, wherein the polymeric composition comprises:
(a) from about 25 wt % to about 30 wt % of sodium polystyrene sulfonate;
(b) from about 8 wt % to about 12 wt % of polyphosphorylcholine butylmethacrylate copolymer;
(c) from about 0.3 wt % to about 0.8 wt % of a preservative; and
(d) from about 55 wt % to about 70 wt % of water.

16. The cosmetic device of claim 12, wherein said housing further comprises a second compartment filled with a cosmetic composition comprising at least one active ingredient capable of biologically stimulating collagen synthesis in skin cells and reducing the appearance of fine lines and wrinkles on the skin, and wherein said device further comprises a second applicator head located at the other, opposite end of said housing and in fluid communication with said second compartment, wherein said second applicator head is arranged and constructed to dispense the cosmetic composition onto a skin surface in form of a line having a width ranging from about 0.1 mm to about 2 mm.

17. The cosmetic device of claim 16, wherein the at least one active ingredient is selected from the group consisting of vitamins A, C, and E and analogues and derivatives thereof, aminoethyl compound, glucans, growth factors, ginsenoside, hydrolyzed proteins, peptide substances or structures and derivatives thereof, collagen fragments, asiatic acid, madecassic acid, madecassoside, asiaticoside, plant extracts from Aloe, Centella, and Plantago species, soy extract and soy isoflavones, extracts from Ginkgo biloba, niacinamide, astaxanthine, genistein, daidzein, rutin, chrysin, morin, betel nut alkaloids, forskolin, betulinic acid, glutamine, glycolic acid, flavonoids, tannins, saponins, resveratrol and derivatives thereof, water-soluble salts of aluminum, calcium, magnesium, and iron, and mixtures thereof.

18. A topical composition comprising:
(a) from about 20 wt % to about 40 wt % of a first polymer selected from the group consisting of sodium polystyrene sulfonate, styrene/acrylates/ammonium methacrylate copolymer, vinyl caprolactam/vinylpyrrolidone/dimethylaminoethylmethacrylate copolymer, vinylpyrrolidone/dimethylaminopropylmethacrylamide copolymer, vinylpyrrolidone/dimethylaminoethylmethacrylate copolymer, and dimethylacrylamide/acrylic acid/polystyrene ethyl methacrylate copolymer;
(b) from about 1 wt % to about 20 wt % of a second polymer selected from the group consisting of polyphosphorylcholine butylmethacrylate copolymer, vinylpyrrolidone/dimethylaminopropylmethacrylate/methylaminopropyldimethyl methacrylate copolymer, vinylpyrrolidone/methacrylamidopropyl trimethylammonium chloride copolymer, vinylpyrrolidone/dimethylaminoethyl methacrylate copolymer, and quaternized hydroxyethylcellulose/hyaluronic acid complex;
(c) from about 0.1 wt % to about 1 wt % of a preservative; and
(d) from about 40 wt % to about 80 wt % of water.

19. The topical composition of claim 18, wherein the first polymer is sodium polystyrene sulfonate, wherein the second polymer is polyphosphorylcholine butylmethacrylate copolymer, and wherein the preservative is phenoxyethanol.

20. The topical composition of claim 19, consisting essentially of sodium polystyrene sulfonate, polyphosphorylcholine butylmethacrylate copolymer, phenoxyethanol, and water.
